(19) United States
(12) Patent Application Publication
(10) Pub. No.: US 2002/0142983 A1
(43) Pub. Date: Oct. 3, 2002

(54) MUC-1 antagonists and methods of treating immune disorders

(75) Inventors: Babita Agrawal, Edmonton (CA); B. Michael Longenecker, Edmonton (CA)

Correspondence Address: Bernhard D. Saxe, FOLEY & LARDNER, Washington Harbour, 3000 K Street, N.W., Suite 500, Washington, DC, 20007-5109, US

(73) Assignee: BIOMIRA, INC.

(21) Appl. No.: 09/984,183

(22) Filed: Oct. 29, 2001

Related U.S. Application Data
(63) Continuation of application No. 09/457,354, filed on Dec. 9, 1999, now abandoned.
(60) Provisional application No. 60/111,973, filed on Dec. 11, 1998.

(51) Int. Cl. A61K 48/00; A61K 31/66; A61K 31/56; A61K 38/13; A61K 31/708

(52) U.S. Cl. 514/44; 514/45; 514/8; 514/251; 514/179; 514/110

(57) ABSTRACT

The invention provides compounds and compositions of containing intracellular inhibitors of the mucin MUC-1. These intracellular MUC-1 inhibitors are exemplified by protein-based inhibitors that contain a targeting and/or an internalization domain, and by antisense nucleic acids. These inhibitors are useful in methods of treating autoimmune disorders.

### BACKGROUND OF THE INVENTION

Mucins are large (>200 kDa) glycoproteins with a high carbohydrate content (50-90% by weight) expressed by a variety of normal and malignant epithelial cells (Strous et al., Crit. Rev. Biochem. Mol. Biol. 27:57 (1992); Devine et al., BioEssays 14:619 (1992)). Among the human mucins, MUC-1 is unique in its cell surface transmembrane expression (Gendler et al., J. Biol. Chem. 265:15286 (1990); Siddiqui et al. Proc. Natl. Acad. Sci. USA 85:2320 (1988); Gendler et al., Proc. Natl. Acad. Sci. USA 84:6060 (1987); Ligtenberg et al., J. Biol. Chem. 265:5573 (1990)).

MUC-1 mucin contains a polypeptide core consisting of 30-100 repeats of a 20 amino acid sequence (Gendler et al., J. Biol. Chem. 265:15286 (1990). The presence of large amounts of oligosaccharides attached along the length of the polypeptide core of MUC-1 mucin enhances its rigidity, resulting in large flexible rod-like molecules that may extend several hundred nanometers from the apical epithelial cell surface into the lumens of ducts and glands (Bramwell et al., J. Cell Sci. 86:249 (1986)).

Adenocarcinoma patients with elevated serum MUC-1 mucin levels have higher numbers of T-cells expressing CD69, an early activation marker, than the patients with normal serum MUC-1 levels (Reddish et al., Cancer Immunol. Immunother. 42:303 (1996); Bowen-Yacyshyn et al., Int. J. Cancer 61:470 (1995). It was hypothesized that patients with high serum MUC-1 levels and high numbers of CD69+peripheral blood T-lymphocytes were in a state of T-cell anergy (Reddish et al., Cancer Immunol. Immunother. 42:303 (1996)) similar to tumor infiltrating lymphocytes (TILs), which are CD69+but appear to be “frozen” in an early activation state and unable to express normal interleukin-2 (IL-2) and IL-2R levels (Alexander et al., J. Immunother. 17:39 (1995); Berd et al., Cancer Immunol. Immunother. 39:141 (1994); Barnd et al., Proc. Natl. Acad. Sci. USA 86:7159 (1989).

Elevated levels of serum MUC-1 are associated with poor survival and a lower anti-cancer immune response of metastatic breast, colorectal and ovarian cancer patients following immunotherapy (Bowen-Yacyshyn et al., 1995 Int. J. Cancer 61:470; MacLean et al., J. Immunother. 20:70 (1997)). Cumulatively, all of these results are consistent with an immunosuppressive role for MUC-1 mucin.

Direct demonstration of an immunosuppressive role of cancer associated MUC-1 mucin came from recent work (Agrawal et al., Nature Med. 4:43 (1998)) showing that cancer associated, affinity purified, MUC-1 mucin and synthetic tandem repeats of MUC-1 polypeptide core inhibited human T-cell proliferative responses to polyclonal stimuli. The degree of inhibition of T-cell proliferation was directly proportional to the number of tandem repeats present on MUC-1 polypeptide core synthetic peptides.

This inhibition was reversible by adding a 16 mer (<1 tandem repeat of the polypeptide core) MUC-1 synthetic peptide (Agrawal et al., Nature Med. 4:43), which confirms the role of the MUC-1 polypeptide core in the inhibition of T-cell responses and suggests an inhibitory mechanism, which involves cross-linking of a T-cell surface molecule. The observation that addition of exogenous interleukin-2 (IL-2) or anti-CD28 monoclonal antibody (mAb) reversed the cancer associated MUC-1 mucin induced inhibition of T-cell response is consistent with the mechanism of inhibition being anergy (Agrawal et al., Nature Med. 4:43). Our understanding of the immunoregulatory role of cancer associated MUC-1 mucin has revealed some of the intricate mechanisms tumor cells use to regulate immune responses for their enhanced survival.

Aside from direct immunomodulatory functions, other functions have been proposed for MUC-1 mucin (Gendler et al., Ann. Rev. Physiol. 57:607 (1995)) which involve steric hindrance by the large glycosylated extracellular domain of cell-cell or cell-substratum interactions, remodeling the cytoskeletal network, or by down-regulating the activities of other molecules such as catenins, cadherins or integrins via signal transduction events (Yamamoto et al., J. Biol. Chem. 272:12492 (1997); Parry et al., Exp. Cell Res. 188:302 (1990). Its cytoplasmic tail is phosphorylated consistent with a transmembrane signal transduction function for MUC-1 (Pandey et al., Cancer Res. 55:40003 (1995); Zrihan-Licht et al., FEBS Lett. 356:130 (1994); Mockensturm-Gardner et al., Mol. Biol. Cell 7:434a (1996); Mockensturm-Gardner et al,, Proc. Amer. Assn. Cancer Res. 39:375a (1998).

Paradoxically, in previous studies MUC-I mucin has been proposed to act both as an anti-adhesive as well as an adhesive molecule. The extended conformation of the extracellular domain of MUC-1 mucin may contribute to the anti-adhesive properties, resulting in reduced cell-cell aggregation and decreased adherence to extracellular matrix components in in vitro adhesion assays (Ligtenberg et al., 1992 Cancer Res. 52:2318; Wesseling et al., 1995 J. Cell Biol. 129:255; Wesseling et al., 1996 Mol. Biol. Cell 7:565). Thus, MUC-1 mucin may protect cancer cells from destruction by natural killer or other immune cells (Hayes et al., 1990 J. Immunol. 145:962; Ogata et al., 1992 Cancer Res. 52:4741; Zhang et al., 1997 Cell. Immunol. 66:158; van de Wiel-van Kemenade et al., 1993 J. Immunol. 151:767).

MUC-1 on cancer cells can also have adhesive features as it expresses carbohydrate structures that may be ligands for selectin-like molecules on endothelial cells (Baeckstrom et al., 1991 J. Biol. Chem. 266:21537; Hanski et al., 1993 Cancer Res. 53:4082; Sikut et al., 1996 Int. J. Cancer 66:617; Zhang et al., 1997 Tumor Biol. 18:175; Zhang et al., 1996 J. Cell. Biochem. 60:538). MUC-1 mucin has also been shown to be a ligand for ICAM-1 (Regimbald et al., 1996 Cancer Res. 56:4244), another adhesion molecule involved in cell-cell interactions. MUC-1 can be shed from tumors and detected in serum (Hayes et al., 1985 J. Clin. Invest. 75:1671; Burchell et al., 1984 Int. J. Cancer 34:763; Boshell et al., 1992 Biochem. Biophys. Res. Commun. 185:1; Williams et al., 1990 Biochem. Biophys. Res. Commun. 170:1331). The presence of soluble MUC-1 has been shown to inhibit adhesive interactions of migrating cells with endothelial cells (Zhang et al., 1997 Tumor Biol. 18:175) and thus could cause decreased recruitment of inflammatory cells to the tumor site.

Although it has primarily been studied based on its association with cancer, MUC-1 is in fact expressed by a variety of normal tissues. A number of secretory epithelial cells, for example, express and secrete MUC-1 mucin. However, this MUC-1 is highly glycosylated, and is therefore somewhat different than cancer-associated MUC-1, which is under-glycosylated.

Various glycoforms of MUC-1 mucin (similar to those of cancer associated MUC-1 mucin) have been found to be present in endometrium and in the serum of pregnant women. McGuckin et al., Tumour Biol. 15:33 (1994). During the menstrual cycle, the abundance of MUC-1 varies in human endometrium. Moreover, progesterone up regulates the transcription of MUC-1 and maximum MUC-1 expression appears in the implantation phase. Hey et al., J. Clin. Endocrinol. Metab. 78:337 (1994).

Interestingly, it has been shown that high levels of progesterone present during days 14-28 of the menstrual cycle are associated with inhibition of cytotoxic T-lymphocyte (CTL) activity in the uterus. Consequently, the down-regulation of CTL activity may allow implantation of a semi-allogeneic embryo, which would be otherwise be rejected. White et al., J. Immunol. 158:3017 (1997). The mechanism of this T-cell down-regulation, however, is unknown. Indeed, the art is generally deficient in its knowledge regarding T-cell activation and de-activation.

T-cell activation is an indicator of the immune state and thus is useful in monitoring a variety of diseases. For example, certain autoimmune diseases are etiologically linked to T-cell activation. Moreover, the ability to control the state of T-cell activation would, likewise, be useful in treating a wide variety of disorders. Autoimmune disorders, for example, represent a diverse collection of disorders, unrelated save for their common inflammatory etiology. T-cell activation is often a key link in this etiology.

Current treatments focus on this etiology and utilize a wide variety of medicaments, including non-steroidal antiinflammatories, corticosteroids, and even cytoablative agents. Unfortunately, neither the existing medicaments nor treatments which utilize them are wholly satisfactory. Likewise, similar dissatisfaction exists with respect to many inflammatory disorders, organ transplant rejection and graft-versus host disease. Thus, there exists a need for new medicaments and new methods of treatment for these disorders.

A need exists, therefore, in the art for the elucidation of a fundamental pathway involved in the regulation of T-cell activation. Provided such a pathway, certain diagnostic and medicinal agents will be made available to the art. The present invention, as detailed below, describes such a novel fundamental pathway as well as a variety of compounds for modulating that pathway, which have certain diagnostic and therapeutic applications.

### SUMMARY OF THE INVENTION

It is, therefore, an object of the invention to provide methods for inducing, preferably T-cell-based, immunosuppression. According to this object, methods are provided which entail contacting a T-cell with an agent that inhibits a cellular process associated with MUC-1 expression. In different embodiments, these cellular processes may be, for example, MUC-1 transcription, MUC-1 translation or MUC-1 protein transport.

It is another object of the invention to provide methods for treating, preferably T-cell-based, autoimmune disorders. According to this object, methods are provided which entail administering to a patient an agent that inhibits a cellular process associated with MUC-1 expression. In different embodiments, these cellular processes may be, for example, MUC-1 transcription, MUC-1 translation or MUC-1 protein transport.

It is still another object of the invention to provide methods for treating, preferably T-cell-based, immune disorders. According to this object, methods are provided which entail administering to a patient an agent that inhibits a cellular process associated with MUC-1 expression. In different embodiments, these cellular processes may be, for example, MUC-1 transcription, MUC-1 translation or MUC-1 protein transport.

It is yet another object of the present invention to provide new methods for treating autoimmune disorders, inflammatory disorders, organ transplant rejection and graft versus host disease. According to this object, methods are provided which comprise administering a pharmaceutically effective amount of intracellular MUC-1 antagonists to a patient in need of said treatment.

It is a further object of the invention to provide novel medicaments to implement methods for treating autoimmune disorders, inflammatory disorders, organ transplant rejection and graft versus host disease. According to this object of the invention compounds and pharmaceutical compositions are provided which comprise an antagonist of MUC-1 function, associated with a domain selected from the group consisting of a targeting domain, an internalization domain and combinations thereof.

### BRIEF DESCRIPTION OF THE DRAWINGS

FIG. 1A shows fluorescence activated cell sorting (FACS) analysis of a time course of MUC-1 expression on activated human T-cells in the absence of mitogen stimulus. The number in parentheses represents percent MUC-1 positive T-cells.

FIG. 1B shows a FACS analysis of a time course of MUC-1 expression on activated human T-cells cultured in the presence of phytohemaglutanin (PHA). The number in parentheses represents percent MUC-1 positive T-cells.

FIG. 2 demonstrates that expression of MUC-1 mucin on T-cells is reversible, as measured by MUC-1-specific antibody. Squares: Peripheral blood lymphocytes (PBLS) were cultured in the presence of PHA for 1, 3 and 6 days. At day 6, the cells were washed, harvested and recultured in the absence of PHA (media alone) for a further 3-6 days. Circles: PBLs were cultured in the absence of PHA for 6 days after which PHA was added and cells were cultured again for a further 6 days.

FIG. 3 demonstrates that antibody cross-linking MUC-1 on the surface of the T-cells modulates proliferative response.

### DETAILED DESCRIPTION OF THE INVENTION

### Overview

The present invention derives from the surprising observation that MUC-1, which heretofore was thought to be biologically important only in the context of certain disease states, plays a key role in the normal immunological response. Thus, when peripheral T-cells isolated from normal human serum, i.e., from non-cancerous patients, were monitored for the presence of MUC-1, only about 3-4% of these were found to express MUC-1. In contrast, upon mitogenic stimulation, as seen in the Examples below, approximately 80% of this same population of T-cells expressed MUC-1. This clearly shows a correlation between T-cell activation and MUC-1.

As set out in detail below, we conclude that MUC-1 mucin is involved in normal immune regulation, more specifically in T-cell activation/inactivation. Evidence supporting this conclusion includes: [1] newly synthesized MUC-1 mucin is rapidly induced and appears on the cell surface of the majority of activated human T-cells; [2] the down-regulation of MUC-1 mucin expression after the mitogenic stimulus is removed; [3] anti-MUC-1 mAb B27.29 (MUC-1-specific) modulates the T-cell proliferative response; [4] new expression of MUC-1; [5] MUC-1 mucin is either shed or secreted into the supernatants of cultures of phytohemaglutanin (PHA) activated human T-cells; [6] soluble MUC-1 mucin inhibits T-cell proliferation and induces an anergy-like state that is reversible by IL-2 or anti-CD28 antibody (Agrawal et al., Nature Med. 4:43 (1998)); and [7] antisense inhibitors of MUC-1 prevent T-cell activation.

This conclusion also explains certain observations from the art that suggest normal functions for MUC-1. Specifically, it unifies the observations that: certain endometrial MUC-1 glycoforms vary during the menstrual cycle; progesterone up-regulates the transcription of MUC-1, maximally during implantation; and the association of high levels of progesterone during days 14-28 of the menstrual cycle with inhibition of cytotoxic T-cell (CTL) activity in the uterus. Since extracellular MUC-1 is herein shown to be a negative regulator of normal T-cell activation, it is likely that MUC-1 is acting to down-regulate CTL activity which would otherwise prevent embryo implantation through CTL-mediated rejection.

In sum, the observation that cancer-associated MUC-1 mucin inhibits human T-cell proliferative response (Agrawal et al., Nature Med. 4:43 (1998)) and the data presented below, showing that MUC-1 mucin is transiently expressed on, and shed or secreted by, activated human T-cells, clearly indicate that MUC-1 mucin plays an important regulatory role in an immune response. In addition, the observations that MUC-1 mucin can present multiple functional domains e.g. anti-adhesion, pro-adhesion as well as inhibit T-cell proliferative response (Agrawal, Nature Med. 4:43 (1998); Ligtenberg et al., Cancer Res. 52:2318 (1992); Wesseling et al., J. Cell Biol. 129:255 (1995); Wesseling et al., Mol. Biol. Cell 7:565 (1996)), are further consistent with the present conclusion that MUC-1 expression on T-cells plays an important homeostatic function. It is likely that MUC-1 mucin on the surface of activated T-cells actively terminates T-cell responses by down regulating their proliferation and, moreover, MUC-1 may serve a role in lymphocyte trafficking due to its adhesion and/or anti-adhesion properties.

Furthermore, it is likely that MUC-1 and/or MUC-1 expression inside the cell induces T-cell activation. Thus, MUC-1 probably works as a timer of T-cell activation. Intracellular MUC-1-associated events induce activation and extracellular MUC-1 acts as a down-regulator of these very same events.

It is known that both inflammatory and autoimmune disorders are associated with a hyper-reactive, or over-reactive, immune response. Due to the involvement of activated T-cells in such illegitimate immune responses, the present invention relates to the use of intracellular MUC-1 antagonists to suppress or prevent that response. This translates to such practical applications as suppressing or preventing transplant rejection and graft versus host reactions. Intracellular MUC-1 antagonists may be employed as immunosuppressive agents to treat these disorders by suppressing the over-reactive immune response. Moreover, these compounds may be employed as commercial reagents for in vitro surrogate systems for T-cell activation/de-activation.

### Definitions

As used in this specification, an “activated T-cell” is one that is in the following phases of the cell cycle: the G₁ phase, the S phase, the G₂ phase or the M (mitosis) phase. Thus, an “activated T-cell” is undergoing mitosis and/or cell division. An activated T-cell may be a T helper (T_{H}) cell or a cytotoxic T-cell (cytotoxic T lymphocyte (CTL or T_{c}n)). Activation of a naive T-cell is initiated, for example, by exposure of such a cell to an antigen presenting cell (APC) (which contains antigen/MHC complexes) and to a molecule such as IL-1. The antigen/MHC complex interacts with a receptor on the surface of the T-cell (T-cell receptor (TCR)). Golub et al., eds. IMMUNOLOGY: A SYNTHESIS, Chapter 2: “The T-cell Receptor” (1991). The skilled artisan will recognize that suitable accessory molecules may also be involved in activation of T-cells. Examples of such accessory molecules include B7.1 (binds to CD28); B7.2 (binds to CTLA4); and intracellular adhesion molecule-1 (ICAM-1; binds to LFA-1).

As used herein, the terms “anergy” and “immunosuppression” are used interchangeably and specifically incorporate all attributes ascribed to these terms, individually and collectively, by the immunological arts. These terms specifically encompass preventing or reversing the cell surface localization on T-cells of MUC-1 and CD25, regardless of whether other indicia of immunosuppression are present, but typically other such indicia are present.

MUC-1 “antagonists” and “inhibitors” are synonymous and, as used generically herein in reference to immunosuppressive methods, they refer to compounds that can act intracellularly; they specifically include intracellular inhibitors or antagonists of MUC-1 expression (protein or mRNA), transport or function. Unless otherwise indicated, the compounds of the invention, as specifically claimed below, however, are not limited to intracellular localisation or action.

The term “treating” in its various grammatical forms in relation to the present invention refers to preventing, curing, reversing, attenuating, alleviating, minimizing, suppressing or halting the deleterious effects of a disease state, disease progression, disease causative agent or other abnormal condition.

In reference to a “sample” from a patient, the term “providing” includes any act of possessing, including obtaining the sample.

As used herein, an “inflammatory disorder” refers to any of the many inflammatory disorders that are well known to those of skill in the art. These disorders include, but are not limited to, the following disorders: inflammatory arthritis such as rheumatoid arthritis, psoriasis, allergies such as allergic contact dermatitis, and ankylosing spondylitis.

As used herein, an “autoimmune disorder” refers to any of the many autoimmune disorders that are well known to those of skill in the art. These disorders include, but are not limited to, the following disorders: myasthenia gravis, systemic lupus erythematosus, polyarteritis nodosa, Goodpastures syndrome, isopathic thrombocytopenic purpura, autoimmune hemolytic anemia, Grave's disease, rheumatic fever, pernicious anemia, insulin-resistant diabetes mellitus, bullous pemphigold, pemphigus vulgaris, viral myocarditis (Cocksakie B virus response), autoimmune thyroiditis (Hashimoto's disease), male infertility (autoimmune), sarcoidosis, allergic encephalomyelitis, multiple sclerosis, Sjorgens disease, Reiter's disease, Celiac disease, sympathetic ophthalmia, and primary biliary cirrhosis.

Unless otherwise indicated by context, the term “RGD” refers not only to the peptide sequence Arg-Gly-Asp, it refers generically to the class of minimal or core peptide sequences that mediate specific interaction with integrins. Thus, an “RDG targeting sequence” encompasses the entire genus of integrin-binding domains.

### Therapeutic Rationale

Due to the correlation of MUC-1 with T-cell activation in normal patients, it is likely that there is a cause-effect relationship. In other words, inhibiting MUC-1 function or expression will at least qualitatively, if not quantitatively, alter T-cell activation. In particular, it is likely that MUC-1 acts as a sort of timer by which the window of T-cell activation is measured. In this way, surface MUC-1 may act by a negative feedback mechanism to transition from an activated state to resting status. On the other hand MUC-1, or MUC-1 expression, inside the cell may be involved in T-cell activation. This hypothesis is consistent with the dual observations that full-length extracellular MUC-1 is immunosuppressive and MUC-1 antisense inhibits T-cell activation.

It is presently demonstrated that when T-cells are stimulated, MUC-1 is expressed, transported to the outer surface of the cell and, to some extent, secreted, i.e., liberated from the cell surface. Once outside the cell, MUC-1 is in a position to interact with other molecules on the T-cell surface. As MUC-1 accumulates on the surface, in the manner analogous to exogenously added MUC-1, it may progressively down-regulate the T-cell response and/or induce T-cell anergy. Hence, for example, it is possible that MUC-1 is responsible for inducing T-cells to transition from an activated state to helper status, where they can be reactivated upon antigenic re-stimulation. In other words, it is likely that MUC-1 plays a normal role in T-cell deactivation, and that this function is usurped by MUC-1-associated tumors to suppress the immune response in general or in particular against them.

On the other hand when normal T-cells are treated to MUC-1-specific antisense molecules, both MUC-1 and CD25 are absent from the surface. Additionally, the cells do not appear activated when cell size is assayed using fluorescence-activated cell sorting (FACS). In other words, these data strongly suggest that intracellular MUC-1 inhibition is immunosuppressive, and thus intracellular MUC-1 or MUC-1 expression has a positive effect on T-cell activation. Accordingly, it is believed that antagonizing the intracellular effects of MUC-1 will be useful in treating diseases associated with illegitimate T-cell activation.

MUC-1 is comprised of many small “core repeats” which are believed to mediate its immunomodulatory effects. MUC-1 derivatives bearing small numbers (<3) of, or individual core repeats have the ability to reverse MUC-1-mediated immunosuppression. Accordingly, MUC-1 probably mediates its effects by crosslinking various surface ligands, a hypothesis supported by FIG. 4, which shows that artificially inducing MUC-1 crosslinking with the aid of an antibody partially abrogates the T-cell response.

The kinetics of MUC-1 induction, relative to other T-cell activation markers, is also suggestive of these possible roles for MUC-1. As demonstrated below in the Examples, relatively small numbers of CD69+and CD25+(markers of T-cell activation) cells express MUC-1 at 24 hours (14.75% and 17.4% respectively). This number, however, increases over the ensuing 5 days to substantial levels (81.6% and 80.2%, respectively). Hence, MUC-1 expression is induced over a relatively long period of time in populations of activated T-cells. These data are clearly consistent with MUC-1 acting as a clock, signalling the duration of the T-cell response, inducing and then actively down-regulating it.

Informed by these data, methods of modulating MUC-1 expression in a T-cell are provided. These methods usually involve contacting a T-cell with an agent that inhibits a cellular process selected from the group consisting of MUC-1 transcription, MUC-1 translation, MUC-1 function and MUC-1 protein transport. They may be implemented using, for example, systemic administration or ex vivo treatment.

### MUC-1-Based Immunosuppressants

A. Intracellular Antagonists of MUC-1 Function

Intracellular MUC-1 antagonists are generally comprised of at least two functional domains. The first domain acts to target the molecule to a cell of interest, typically a T-cell, and/or to induce cellular internalization of the molecule. The second domain functions as an antagonist of MUC-1 function. As explained below, the targeting and internalization functions may reside together in one molecule or in two separate molecules.

1. Intracellular Localization

Since each of the following MUC-1 derivatives and inhibitors is intended for intracellular use, they are preferably modified in a manner to facilitate intracellular localization. One specifically contemplated method is modification with a targeting domain (targeting signal) that directs any associated molecule to the external cell membrane. This can be accomplished by coupling any of the therapeutic molecules discussed below to a targeting domain. These targeting domains may be relatively large molecules, such as antibodies (e.g., directed to CD3), but they are preferably small, like Fab molecules. Even more preferably, these targeting domains are small peptides, for example, less than about 20 amino acids. The size, however, is important only in that the smaller molecules will typically have a greater likelihood of intracellular localisation.

Directing a molecule to the surface of the cell is known to facilitate uptake of the molecule, presumably through endocytic means. See, for example, Hart et al., J. Biol. Chem. 269:12468-74 (1994) (internalisation of phage bearing RGD); Goldman et al, Gene Ther. 3:811-18 (1996) (RGD-mediated adenoviral infection) and Hart et al., Gene Ther. 4:1225-30 (1997) (RGD-mediated transfection). Thus, a targeting domain in many cases will act as an internalization domain, as well.

Many such targeting signals are known in the art. One class of targeting signals, which bind specifically to integrins (points of extracellular matrix attachment), bears a the peptide signal sequence based on Arg-Gly-Asp (RGD). Yet another class includes peptides having a core of Ile-Lys-Val-Ala-Val (IKVAV). See Weeks et al., Cell Inmunol. 153:94-104 (1994). Of course, antibodies or antibody fragments (as described below) may be used to specifically target therapeutic molecules to cell surface markers.

In the case of protein- or peptide-based MUC-1 derivatives and inhibitors, these targeting signals may be engineered directly into any expression system or added in any peptide synthesis, thereby forming an intracellular MUC-1 inhibitor. A targeting signal may be added at the N- or the C-terminus or both.

For peptide and non-peptide-based MUC-1 derivatives and inhibitors, a targeting signal may be added chemically. Many commercially available cross-linkers are suitable for this purpose. Typically these crosslinkers require free thiol (e.g., maleimide-based) or amino groups (e.g., succinimide-based) with which to react. Hence, the addition of amino acids such as cysteine, methionine, arginine and lysine is contemplated to facilitate this process. For antisense molecules and other nucleic acid-based approaches, targeting sequences may be attached to polyamines, which then can be complexed to the nucleic acid for efficient delivery. A preferred approach uses a targeting sequence, like RGD, coupled to polylysine, which may be ionically complexed with a suitable nucleic acid.

Integrins are an especially suitable target for the present inventive compounds, because increased integrin-binding, likely due to up-regulation of integrins, is associated with T-cell activation. See Weeks et al. (1994), supra. Since the present compounds are generally immunosuppressive, and exert this effect against T-cells, such a targeting mechanism will direct the present therapeutic compounds to their intended target at precisely the right time. In other words, the inventive compounds will be directed preferentially to the T-cells when they are activated, thereby inducing de-activation and preventing re-activation, i.e. immunosuppression and/or anergy will result. Thus, the paradigm RGD-based targeting sequences are contemplated.

Some, particularly preferred integrin targeting sequences may be found in U.S. Pat. Nos. 5,041,380 (1991), 5,591,592 (1997), 5,622,699 (1997) and 5,627,263 (1997), the sequences of which are hereby incorporated by reference. In addition, U.S. Pat. Nos. 5,591,592 (1997) and 5,622,699 (1997) may be consulted for methods of deriving additional integrin-binding sequences that are more particularly directed to lymphocytes, which are a contemplated therapeutic target of the present invention. Similar methods are disclosed in Koivunen et al., J. Biol. Chem. 268:20205-10 (1993).

It is also contemplated that a non-structural spacer may be placed between the MUC-1 derivative proper and the targeting domain. Such spacers typically comprise glycine and/or proline residues. Preferably lengths of these spacers range from about one to about 5 amino acids, with two being particularly preferred. In addition, it is often preferable to physically constrain the targeting domain by cyclization, which usually results in increased binding. This is usually accomplished by a pair of cysteine residues, flanking the RGD core at a distance of about 4 (having only RGD in between) to 10 amino acids from one another, and preferably about 7 amino acids from one another.

Thus, a typical targeting domain would have the following structure:

-XRGDYX-

wherein X is zero to five amino acids and Y is a one or two amino acids, selected from cysteine, serine, threonine and methionine. In a particularly useful embodiment, X is comprised of glycine residues, but optionally contains at least one, and typically one or two, free thiol- or amine-containing amino acids and/or a single hydrophobic amino acid. Thiol-containing residues include methionine and cysteine; amine-containing residues include lysine and (at least one additional) arginine; and hydrophobic residues include leucine, isoleucine, alanine and phenylalanine.

In order to improve the intracellular localization of the present intracellular inhibitors, a preferred approach uses, either alone or in conjunction with a targeting domain, an internalization domain, such as a retrograde transport sequence. Retrograde transport sequences derive from proteins that are able to move from outside of the cell to the inside, against the normal protein trafficking mechanisms of the cell. See Wiedlocha, Arch. Immunol. Ther. Exp. 44:201-07 (1996) for a review. The paradigm may be derived from examples that include fibroblast growth factor (both acidic and basic), interleukin 1, angiogenin, Schwannoma-derived growth factor, the Antennapedia homeoprotein and HIV-1 Tat. A specific example is the peptide Lys-Asp-Glu-Leu (KDEL), which normally functions as an intracellular retention signal, but can also mediate retrograde transport. Johannes et al., J. Biol. Chem. 272:19554-61 (1997).

A preferred approach utilizes the protein transduction domain (PTD) of the HIV tat protein as an internalization domain. While its mechanism of action is unknown, this sequence appears to act in a manner independent of normal cellular transport systems. The protein transduction domain is located between amino acids 49 and 57 of the HIV tat protein, with a preferred sequence comprising the following amino acid sequence: YGRKKRRQRRR. The complete tat seqeunce may be found at GenBank Accession No. P04606, and in Frankel et al., U.S. Pat. No. 5,804,604 (September 8, 1998). Thus, as used herein, the “tat PTD” encompasses the native sequence, as described in the foregoing documents, and it encompasses variants of that sequence that retain the protein translocation activity of the parent molecule.

The tat PTD may be added chemically, as described in the Frankel patent and above. For such purposes it is beneficial to include a cysteine residue in the sequence of the PTD. Alternatively, as described in Schwarze et al., Science 285: 1569-72 (1999), the PTD may be added by construction of a fusion protein/peptide. It is also beneficial to include between the MCU-1 antagonist, or other domain, and the tat PTD, a non-structural linker sequence, which is comprised of at least one proline or glycine residue. Typical linker sequences comprise from one to ten amino acids, but generally will be between two and seven amino acids, or even three to five amino acids.

2. MUC-1 Derivatives

Beneficially, MUC-1 antagonists that contain a PTD will be denatured prior to use. This is described in more detail in Nagahara et al., Nature Medicine 4: 1449-52 (1998), which provides a basic protocol. Denaturation usually comprises contacting the subject antagonist with a denaturant, like a chaotrope (e.g., urea or a guanidium salt—4-8 molar) or a detergent, then removing the denaturant in a manner to maintain the denatured state of the molecule. Removing the denaturant, thus, is done fairly rapidly, for example, by dialysis, ultrafiltration or column chromatography.

Therapeutic compounds that antagonize intracellular MUC-1 function are herein generically termed “MUC-1 derivatives.” The compounds are not limited, however, to those specifically derived from MUC-1, but include the entire class of compounds which exhibit activity in antagonising MUC-1-mediated T-cell activation. Combinations of any of the following permutations are also possible and, to the extent that these combinations fall within the biological and physical description below, they are still considered “MUC-1 derivatives.

An important class of MUC-1 derivatives includes peptide derivatives. Specific peptide-based derivatives include those derived from the sequence of the core repeat of native MUC-1. In one embodiment, the peptide would include the extracellular tandem repeat region of MUC-1, which includes repeats of the amino acid sequence DTRP (Asp-Thr-Arg-Pro). Preferably these tandem repeats include the sequence SAPDTRP (Ser-Ala-Pro-Asp-Thr-Arg-Pro). As modified with targeting signals, these peptides become XRGDYXDTRP, DTRPXRGDYX, XRGDYXSAPDTRP or SAPDTRPXRGDYX.

A MUC-1 “core repeat,” “core sequence” or “MUC-1 core” as used herein generally refers to that present in the native MUC-1 molecule, which comprises the 20 amino acid sequence PDTRPAPGSTAPPAHGVTSA (Pro-Asp-Arg-Thr-Pro-Ala-Pro-Gly-Ser-Thr-Ala-Pro-Pro-Ala-His-Gly-Val-Thr-Ser-Ala), and derivatives of this sequence, such as PDTRPAPGSTAPPAHGVTSAXRGDYX and XRGDYXPDTRPAPGSTAPPAHGVTSA. Thus, different permutations of the 20 amino acid core sequence may be used, including substitutions, deletions, other permutations, and multiple repeats of any of the foregoing. For example, conserving the basic amino acid order and size of the peptide, the starting residue may be permuted. In one example, the repeat may begin with GVTSA, instead of PDTRP, for example, yielding GVTSAPDTRPAPGSTAPPAH. Other, similar permutations are also possible where the single repeat is linearly permuted by simply beginning with a different amino acid.

Deletion derivatives, including truncations and internal deletions, are especially useful. One particularly useful MUC-1 derivative of this class is a 16 amino acid peptide of the sequence GVTSAPDTRPAPGSTA. Containing a targeting sequence, this peptide becomes GVTSAPDTRPAPGSTAXRGDYX or XRGDYXGVTSAPDTRPAPGSTA.

Some preferred peptide-based MUC-1 derivatives comprise one, or less than one, peptide core repeat of the MUC-1 mucin. A recitation of “at most one MUC-1 core repeat” contemplates a minimum of about 6 amino acids and even more preferably at least about ten. This, of course, is subject to such a molecule having the requisite T-cell activation-suppressing properties. The maximum size of “at most one MUC-1 core repeat” would be 20 amino acids, as prescribed by the native length. Hence a preferred length is about ten to about twenty amino acids.

Further MUC-1 derivatives include modified versions of a single MUC-1 core repeat. For example, given the basic repeat sequence, conservative substitutions may be made which preserve the requisite anergy/immunosuppression-reversing characteristics. Amino acid substitutions, i.e. “conservative substitutions,” may be made, for instance, on the basis of similarity in polarity, charge, solubility, hydrophobicity, hydrophilicity, and/or the amphipathic nature of the residues involved.

For example: (a) nonpolar (hydrophobic) amino acids include alanine, leucine, isoleucine, valine, proline, phenylalanine, tryptophan, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in a-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

Other substitutions include replacing the L-amino acid with the corresponding D-amino acid. This rationale, moreover can be combined with the foregoing conservative substitution rationales. For example, D-serine may be substituted for L-threonine. In addition, these D-amino acid-containing peptides may be prepared which have an inverse sequence, relative to the native sequence. Hence, DTRP becomes PRTD. Such “retro-inverso” peptides are expected to have improved properties, such as increased in vivo half-life. This translates into smaller doses and more economically viable production. Of course, retro-inverso peptides may be prepared with D-amino acids as well.

Other useful MUC-1 derivatives include glycosylated or non-glycosylated peptides. Glycosylation can be biological or non-biological. For example, biologically relevant N- or O-linked carbohydrates are envisioned. Other chemical modifications, such as succinylation are also contemplated. These specifically include modification with polyethylene glycols.

MUC-1 derivatives also specifically include multiple repeats of any of the specific derivatives defined herein. Moreover, each of the foregoing derivatives can be mixed and matched with each other. These multiple repeats are preferably tandem and usually will have a maximum of three repeated units. Thus, for example, a multiple repeat containing the full 20 amino acid core sequence would have a maximum length of 60 amino acids. However, the maximum number of repeated units ultimately will be determined by the ability of the MUC-1 derivative to inhibit T-cell activation.

Although small peptides may be preferable from both economic and certain technical perspectives, larger molecules are also contemplated. Thus, peptide-based MUC-1 derivatives may be combined with other useful therapeutic agents, yielding enhanced properties. They may be so combined, for example, covalently or electrostatically. Ideally these other therapeutic agents will be immunomodulators, and preferably will have immunosuppressive properties. Examples include non-steroidal antiinflammatories, corticosteroids, and even cytoablative agents. Specific examples include azathioprine, chlorambucil, cyclophosphamide, cyclosporine, dactinomycin, methotrexate and thioguanine, dexamethasome, betamethasone, cortisone, hydrocortisone, mycophenolate, and prednisolone.

Specific useful MUC-1 derivatives can be derived from purified MUC-1, or portions thereof, produced by native sources or recombinant DNA methodology, by methods that include digestion with enzymes such as pepsin or papain. Alternatively, peptides encompassed by the present invention can be synthesized using an automated peptide synthesizer such as those supplied commercially by Applied Biosystems, Multiple Peptide Systems and others, or they may be produced manually, using techniques well known in the art. See Geysen et al., J. Immunol. Methods 102: 259 (1978). Glycosylated and other forms of peptide or protein MUC-1 derivatives may be made according to methods well known in the art.

Although preferred MUC-1 derivatives are protein- (or peptide-) based, other derivatives are contemplated. For example, small molecules which are amino acid or peptide mimetics may be useful. Rational design of such molecules is possible using methods known in the art. Using, for example, space-filling models, otherwise structurally unrelated compounds may be made to mimic protein-based MUC-1 derivatives. The usefulness of these MUC-1 derivatives can be confirmed using routine assays, such as those presented in Agrawal et al., Nature Medicine, 4:43 (1998).

Further intracellular MUC-1 antagonists include normal ligands of MUC-1. Especially preferred among these ligands are cell adhesion molecules, such as intracellular adhesion molecule-1 (ICAM-1). In addition, these ligands may be shorter, for example proteolytically or recombinantly produced, truncated versions or fragments. They should, however, retain the ability to inhibit MUC-1-induced T-cell activation. Of course, these typically will be modified with a targeting sequence or otherwise formulated for intracellular delivery.

3. Antibody-Based Intracellular MUC-1 Antagonists

Still another important class of MUC-1 antagonists is antibody-based antagonists. Antibodies raised against MUC-1 and its fragments are specifically contemplated. Antibodies include, but are not limited to polyclonal antibodies, monoclonal antibodies (mAbs), humanized or chimeric antibodies, single chain antibodies including single chain Fv (scFv) fragments, Fab fragments, F(ab′)2 fragments, fragments produced by a Fab expression library, epitope-binding fragments, and humanized forms of any of the above. Of course, the smaller versions of these molecules are preferred, based on the fact that they will more readily localize to the inside of a cell. Again, the same localization signals, detailed above, are useful with this class of MUC-1 antagonist.

In general, techniques for preparing polyclonal and monoclonal antibodies as well as hybridomas capable of producing the desired antibody are well known in the art (Campbell, A. M., Monoclonal Antibody Technology: Laboratory Techniques in Biochemistry and Molecular Biology, Elsevier Science Publishers, Amsterdam, The Netherlands (1984); St. Groth et al., J. Immunol. Methods 35:1-21 (1980); Kohler and Milstein, Nature 256:495-497 (1975)), the trioma technique, the human B-cell hybridoma technique (Kozbor et al., Immunology Today 4:72 (1983); Cole et al., in Monoclonal Antibodies and Cancer Therapy, Alan R. Liss, Inc. (1985), pp. 77-96). Affinity of the antisera for the antigen may be determined by preparing competitive binding curves, as described, for example, by Fisher, Chap. 42 in: Manual of Clinical Immunology, second edition, Rose and Friedman, eds., Amer. Soc. For Microbiology, Washington, D.C. (1980).

Fragments or derivatives of antibodies include any portion of the antibody which is capable of binding MUC-1. Antibody fragments specifically include F(ab′)₂, Fab, Fab′ and Fv fragments. These can be generated from any class of antibody, but typically are made from IgG or IgM. They may be made by conventional recombinant DNA techniques or, using the classical method, by proteolytic digestion with papain or pepsin. See CURRENT PROTOCOLS IN IMMUNOLOGY, chapter 2, Coligan et al., eds., (John Wiley & Sons 1991-92).

F(ab′)2 fragments are typically about 110 kDa (IgG) or about 150 kDa (IgM) and contain two antigen-binding regions, joined at the hinge by disulfide bond(s). Virtually all, if not all, of the Fc is absent in these fragments. Fab′ fragments are typically about 55 kDa (IgG) or about 75 kDa (IgM) and can be formed, for example, by reducing the disulfide bond(s) of an F(ab′)₂ fragment. The resulting free sulfhydryl group(s) may be used to conveniently conjugate Fab′ fragments to other molecules, such as localization signals. Fab fragments are monovalent and usually are about 50 kDa (from any source). Fab fragments include the light (L) and heavy (H) chain, variable (V_{L} and V_{H}, respectively) and constant (C_{L} C_{H=l , respectively) regions of the antigen-binding portion of the antibody. The H and L portions are linked by one or more intramolecular disulfide bridges.} 5 Fv fragments are typically about 25 kDa (regardless of source) and contain the variable regions of both the light and heavy chains (V_{L} and V_{H}, respectively). Usually, the V_{L} and V_{H} chains are held together only by non-covalent interactions and, thus, they readily dissociate. They do, however, have the advantage of small size and they retain the same binding properties of the larger Fab fragments. Accordingly, methods have been developed to crosslink the V_{L} and V_{H} chains, using, for example, glutaraldehyde (or other chemical crosslinkers), intermolecular disulfide bonds (by incorporation of cysteines) and peptide linkers. The resulting Fv is now a single chain (i.e., scFv).

Other antibody derivatives include single chain antibodies (U.S. Pat. No. 4,946,778; Bird, Science 242:423426 (1988); Huston et al., Proc. Natl. Acad. Sci. USA 85:5879-5883 (1988); and Ward et al., Nature 334:544-546 (1989)). Single chain antibodies are formed by linking the heavy and light chain fragments of the Fv region via an amino acid bridge, resulting in a single chain FV (scFv).

Derivatives also include “chimeric antibodies” (Morrison et al., Proc. Natl. Acad. Sci., 81:6851-6855 (1984); Neuberger et al., Nature, 312:604-608 (1984); Takeda et al., Nature, 314:452454 (1985)). These chimeras are made by splicing the DNA encoding a mouse antibody molecule of appropriate specificity with, for instance, DNA encoding a human antibody molecule of appropriate specificity. Thus, a chimeric antibody is a molecule in which different portions are derived from different animal species, such as those having a variable region derived from a murine mAb and a human immunoglobulin constant region. Recombinant molecules having a human framework region and murine complementarity determining regions (CDRs) also are made using well-known techniques. These are also known sometimes as “humanized” antibodies and they and chimeric antibodies or antibody fragments offer the added advantage of at least partial shielding from the human immune system. They are, therefore, particularly useful in therapeutic in vivo applications.

In addition to their use as direct antagonists of MUC-1 function, the MUC-1 antibody fragments may be useful as inhibitors of MUC-1 transport. Thus, in an ex vivo method, a T-cell-containing sample is provided from a patient. The constituent T-cells are then permeablized using known methods and treated with at least one MUC-1 antibody fragment or derivative thereof.

B. Inhibitors of MUC-1 Protein Transport

Inhibitors of protein transport are also useful in the methods herein disclosed. While these inhibitors may be general in nature, e.g., Brefeldin A, preferred inhibitors are MUC-1-specific. Specific inhibitors may be isolated as described below. Where non-specific or less specific inhibitors of MUC-1 transport are used, ex vivo methods will generally be employed so as to avoid possible unwanted side effects. Antibody fragments, described above, are examples of specific inhibitors of MUC-1 protein transport.

C. Antisense Inhibitors

Given the known sequence of the MUC-1 gene (GenBank Accession Numbers M61170, X54350 and X54351), and its associated control elements, certain MUC-1-specific inhibitors of expression may be rationally designed. Most commonly, these inhibitors will be relatively small RNA or DNA molecules because they can be designed to be highly specific. In general, so-called “antisense” molecules will have a sequence which is complementary to a portion of the MUC-1 mRNA, preferably the pre-mRNA, i.e., the pre-splicing version. More preferred antisense molecules will be specific for the 5′ one-third portion of the MUC-1 mRNA. One particularly preferred class of antisense molecules is directed to the control elements for splicing and/or translation. Such “translational control elements” include the very 5′ end of the mRNA (where the ribosome associates with the mRNA) and the translational start site (an ATG, from the non-coding DNA perspective). The “splicing control elements” include the splice junctions. It may also be advantageous to direct antisense molecules to introns themselves, especially those near the 5′ end of the gene.

As indicated, the antisense molecules can have a variety of chemical constitutions, so long as they retain the ability specifically to bind at the indicated control elements. Thus, especially preferred molecules are oligo-DNA, RNA and protein nucleic acids (PNAs). The oligonucleotides of the present invention can be based, for example, upon ribonucleotide or deoxyribonucleotide monomers linked by phosphodiester bonds, or by analogues linked by methyl phosphonate, phosphorothioate, or other bonds. These can be engineered using standard synthetic techniques to very specifically bind the targeted control region(s). While these molecules may also be large, they are preferably relatively small, i.e., corresponding to less than about 50 nucleotides, more preferably less than about 25 nucleotides. Such oligonucleotides may be prepared by methods well-known in the art, for instance using commercially available machines and reagents available from Perkin-Elmer/Applied Biosystems (Foster City, Calif.).

Phosphodiester-linked oligonucleotides are particularly susceptible to the action of nucleases in serum or inside cells, and therefore in a preferred embodiment the oligonucleotides of the present invention are phosphorothioate or methyl phosphonate-linked analogues, which have been shown to be nuclease-resistant. See Stein et al., (1993), supra. Persons knowledgeable in this field will be able to select other linkages for use in the present invention.

The relative activity of antisense oligonucleotides directed against a specific gene is generally inversely proportional to its location relative to the AUG start codon of the target gene. Accordingly, it is preferred that an antisense oligonucleotide targeted at a specific MUC-1 gene sequence be chosen such that the oligonucleotide hybridizes within approximately 25 bases of the AUG start codon of the gene.

To select the preferred length for an antisense oligonucleotide, a balance must be struck to gain the most favorable characteristics. Shorter oligonucleotides 10-15 bases in length readily enter cells, but have lower gene specificity. In contrast, longer oligonucleotides of 20-30 bases offer superior gene specificity, but show decreased kinetics of uptake into cells. See Stein et al., Phosphorothioate Oligodeoxynucleotide Analogues in “Oligodeoxynucleotides—Antisense Inhibitors of Gene Expression” Cohen, Ed. McMillan Press, London (1988). In a preferred embodiment, this invention contemplates using oligonucleotides approximately 14 to 25 nucleotides long.

Antisense molecules can be delivered in a variety of ways. They may be synthesized and delivered as a typical pharmaceutical, usually parenterally. They may be formulated as detailed below, but one preferred formulation involves encapsulation/association with cationic liposomes. They may be modified with a targeting sequence, is optionally linked to a polyamine, such a polylysine, as described above. See Bachmann et al., J. Mol. Med. 76:126-32 (1998) for one approach to delivering antisense molecules using a targeting sequence. Alternatively, antisense molecules may be delivered using gene therapy methods, detailed below. Using gene therapy vectors, single, or multiple tandem copies of antisense molecules can be used.

Administration of an antisense oligonucleotide to a subject can be effected orally or by subcutaneous, intramuscular, intraperitoneal, or intravenous injection. Pharmaceutical compositions of the present invention, however, are advantageously administered in the form of injectable compositions. A typical composition for such purpose comprises a pharmaceutically acceptable solvent or diluent and other suitable, physiologic compounds. For instance, the composition may contain oligonucleotide and about 10 mg of human serum albumin per milliliter of a phosphate buffer containing NaCl.

As much as 700 milligrams of antisense oligodeoxynucleotide has been administered intravenously to a patient over a course of 10 days (i.e., 0.05 mg/kg/hour) without signs of toxicity. Sterling, “Systemic Antisense Treatment Reported,” Genetic Engineering News 12: 1, 28 (1992).

D. Ribozyme Inhibitors of MUC-1

Another nucleic-acid-based method for down-regulating MUC-1 protein expression utilizes “ribozymes.” Ribozymes are small RNA molecules that characteristically bind a specific, complementary RNA sequence (i.e., MUC-1 mRNA) and cleave the bound target at a specific site. Technology for the design and manufacture of ribozymes is known in the art. See, for example, Haseloff et al., U.S. Pat. Nos. 5,574,143 (1996), U.S. Pat. No. 5,589,580 (1996) and U.S. Pat. No. 5,432,508 (1996), and Kramer et al. U.S. Pat. No. 5,616,459 (1997) which are hereby incorporated by reference in their entirety. Gene Therapy Delivery of Antisense and Ribozyme Molecules

Methods of using antisense and ribozyme technology to control gene expression, or of gene therapy methods for expression of an exogenous gene in this manner are well known in the art. These methods may be performed either in vivo or ex vivo. Each of these methods requires a system for introducing a vector into the cells containing the mutated gene. The vector encodes either an antisense or ribozyme transcript complementary to MUC-1-associated sequences. The construction of a suitable vector can be achieved by any of the methods well-known in the art for the insertion of exogenous DNA into a vector. See, e.g., Sambrook et al., Molecular Cloning (Cold Spring Harbor Press 2d ed. 1989), which is incorporated herein by reference. In addition, the prior art teaches various methods of introducing exogenous genes into cells in vivo. See Rosenberg et al., Science 242:1575-1578 (1988) and Wolff et al., PNAS 86:9011-9014 (1989), which are incorporated herein by reference.

The routes of delivery include systemic administration, administration in situ and ex vivo administration, with the latter being preferred. Well-known techniques include administration with cationic liposomes. The use of a cationic liposome, such as DC-Chol/DOPE liposome, has been widely documented as an appropriate vehicle to deliver DNA to a wide range of tissues through intravenous injection of DNA/cationic liposome complexes. See Caplen et al., Nature Med. 1:3946 (1995) and Zhu et al., Science 261:209-211 (1993), which are herein incorporated by reference. Liposomes transfer genes to the target cells by fusing with the plasma membrane. The entry process is relatively efficient, but once inside the cell, the liposome-DNA complex has no inherent mechanism to deliver the DNA to the nucleus. As such, most of the lipid and DNA gets shunted to cytoplasmic waste systems and destroyed. The obvious advantage of liposomes as a gene therapy vector, as opposed to a purely viral system, is that liposomes contain no proteins, which thus minimizes the potential of host immune responses.

As another example, viral vector-mediated gene transfer is also a suitable method for the introduction of the vector into a target cell. Appropriate viral vectors include adenovirus vectors and adeno-associated virus vectors, retrovirus vectors and herpesvirus vectors.

Adenoviruses are linear, double stranded DNA viruses complexed with core proteins and surrounded by capsid proteins. The common serotypes 2 and 5, which are not associated with any human malignancies, are typically the base vectors. By deleting parts of the virus genome and inserting the desired gene under the control of a constitutive viral promoter, the virus becomes a replication deficient vector capable of transferring the exogenous DNA to differentiated, non-proliferating cells. To enter cells, the adenovirus fiber interacts with specific receptors on the cell surface, and the adenovirus surface proteins interact with the cell surface integrins. The virus penton-cell integrin interaction provides the signal that brings the exogenous gene-containing virus into a cytoplasmic endosome. The adenovirus breaks out of the endosome and moves to the nucleus, the viral capsid falls apart, and the exogenous DNA enters the cell nucleus where it functions, in an epichromosomal fashion, to express the exogenous gene. Detailed discussions of the use of adenoviral vectors for gene therapy can be found in Berkner, Biotechniques 6:616-629 (1988) and Trapnell, Advanced Drug Delivery Rev. 12:185-199 (1993), which are herein incorporated by reference. Adenovirus-derived vectors, particularly non-replicative adenovirus vectors, are characterized by their ability to accommodate exogenous DNA of 7.5 kB, relative stability, wide host range, low pathogenicity in man, and high titers (10⁴ to 10⁵ plaque forming units per cell). See Stratford-Perricaudet et al., PNAS 89:2581 (1992).

Adeno-associated virus (AAV) vectors also can be used for the present invention. AAV is a linear single-stranded DNA parvovirus that is endogenous to many mammalian species. AAV has a broad host range despite the limitation that AAV is a defective parvovirus which is dependent totally on either adenovirus or herpesvirus for its reproduction in vivo. The use of AAV as a vector for the introduction into target cells of exogenous DNA is well-known in the art. See, e.g., Lebkowski et al., Mole. & Cell. Biol. 8:3988 (1988), which is incorporated herein by reference. In these vectors, the capsid gene of AAV is replaced by a desired DNA fragment, and transcomplementation of the deleted capsid function is used to create a recombinant virus stock. Upon infection the recombinant virus uncoats in the nucleus and integrates site-specifically into the host genome.

Another suitable virus-based gene delivery mechanism is retroviral vector-mediated gene transfer. In general, retroviral vectors are well-known in the art. See Breakfield et al., Mole. Neuro. Biol. 1:339 (1987) and Shih et al., in VACCINEs 85: 177 (Cold Spring Harbor Press 1985). A variety of retroviral vectors and retroviral vector-producing cell lines can be used for the present invention. Appropriate retroviral vectors include Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus. These vectors include replication-competent and replication-defective retroviral vectors. In addition, amphotropic and xenotropic retroviral vectors can be used. Suitable producer cells for making viral vectors include fibroblasts, neurons, glial cells, keratinocytes, hepatocytes, connective tissue cells, ependymal cells, chromaffin cells. See Wolff et al., PNAS 84:3344 (1989).

A retroviral vector generally is constructed such that the majority of its structural genes are deleted or replaced by exogenous DNA of interest, and such that the likelihood is reduced that viral proteins will be expressed. See Bender et al., J. Virol. 61:1639 (1987) and Armento et al., J. Virol. 61:1647 (1987), which are herein incorporated by reference. To facilitate expression of the antisense or ribozyme molecule, a retroviral vector employed in the present invention must integrate into the genome of the host cell, an event which occurs only in mitotically active cells, such as T-cells. To minimize unwanted delivery and/or integration events, these methods typically would be performed ex vivo and may use a replication deficient virus.

Clinical trials employing retroviral vector therapy treatment have been approved in the United States. See Culver, Clin. Chem. 40: 510 (1994). Retroviral vector-containing cells have been implanted into brain tumors growing in human patients. See Oldfield et al., Hum. Gene Ther. 4: 39 (1993).

Yet another suitable virus-based gene delivery mechanism is herpesvirus vector-mediated gene transfer. While much less is known about the use of herpesvirus vectors, replication-competent HSV-1 viral vectors have been described in the context of antitumor therapy. See Martuza et al., Science 252: 854 (1991).

E. Identifying Inhibitors of MUC-1 Expression and Transport

In addition to the inhibitors detailed above, the artisan will be well equipped to identify and produce additional inhibitors, and especially those for inhibiting MUC-1. With the advent of combinatorial chemistry, the availability of suitable starting material is immense. As known in the art, these methods are amenable to classical small molecule synthesis as well as macromolecule synthesis, including proteins, lipids, nucleic acids and mimetics thereof, such as protein nucleic acids (PNAs). Moreover, the development of high-throughput screening technology has enabled the quite rapid screening and refinement of combinatorial libraries, which results in the routine identification of pharmacologically active candidates using minimal expense, time and experimentation. The identification of further inhibitors of MUC-1 expression and transport, therefore, is dependent only upon the availability of adequate screening technology. The present invention solves this problem by immediately suggesting to the artisan these very assays.

For instance, inhibitors of MUC-1 expression may be screened using intact cells, or a purely cell free system. In either case, this assay can be adapted to high throughput analysis. In a typical method, the MUC-1 transcriptional control elements are cloned into a suitable vector upstream of an indicator gene, such as β-galactosidase (β-gal).

In a cell-based system, the resulting vector could be transferred, either stably or transiently, to a suitable cell line or primary T-cell culture. The cells would be plated to, for example, 96-well tissue culture plates and treated with a suitable test compound in the presence of a MUC-1 stimulus, such as PHA or progesterone. The accumulation of MUC-1-driven β-gal expression would then be monitored using commercially available chromogenic substrates and a standard or automated plate reader. The inhibition of β-gal expression indicates a candidate compound. Candidates may further be confirmed using other standard assays, such as the RT-PCR assay presented below in the Examples. Generally acting transcription inhibitors could readily be excluded based on internal controls, such as a control promoter driving a different indicator gene. A cell-free system would work essentially the same way, except an in vitro transcription system would be used in place of the cells.

### Pharmaceutical Compositions of the Invention

The inventive compositions may be formulated for administration in a variety of ways. The pharmaceutical compositions of the invention generally contain a pharmaceutically effective amount of an inventive compound. Preferably, the compound is admixed with a pharmaceutically effective vehicle (excipient).

A suitable formulation will depend on the nature of the specific medicament chosen, whether the treatment is in vivo or ex vivo, the route of administration desired and the judgement of the attending physician. Suitable formulations and pharmaceutically effective vehicles, can be found, for example, in REMINGTON'S PHARMACEUTICAL SCIENCES, chapters 83-92, pages 1519-1714 (Mack Publishing Company 1990) (Remington's), which are hereby incorporated by reference.

Preferred vehicles include liposomes. See, for example, Remington's at 1691-92. Thus, the inventive compositions may also be formulated, and administered, in combination with other known medicaments, which may provide. complementary anergy/immunosuppression relieving activity, in liposomal formulations. Preferred other medicaments include the immunosuppressants discussed above. When these known medicaments are formulated and/or used with the present MUC-1 inhibitors, guidance on formulations may come from standard texts. Examples include DRUG INFORMATION FOR THE HEALTH CARE PROFESSIONAL, 18^{th} edition, vol. 1 (U.S. Pharmacopeial Convention, Inc. 1998) and GOODMAN AND GILMAN'S: THE PHARMACOLOGICAL BASIS OF THERAPEUTICS (MacMillan Publishing Co. Current Edition).

Techniques for preparation of liposomes and the formulation (e.g., encapsulation) of various molecules, including peptides and oligonucleotides, with liposomes are well known to the skilled artisan. Liposomes are microscopic vesicles that consist of one or more lipid bilayers surrounding aqueous compartments. See, generally, Bakker-Woudenberg et al., Eur. J. Clin. Microbiol. Infect. Dis. 12 (Suppl. 1): S61 (1993) and Kim, Drugs 46: 618 (1993). Liposomes are similar in composition to cellular membranes and as a result, liposomes generally can be administered safely and are biodegradable.

Depending on the method of preparation, liposomes may be unilamellar or multilamellar, and can vary in size with diameters ranging from 0.02 μm to greater than 10 μm. A variety of agents can be encapsulated in liposomes. Hydrophobic agents partition in the bilayers and hydrophilic agents partition within the inner aqueous space(s). See, for example, Machy et al., LIPOSOMES IN CELL BIOLOGY AND PHARMACOLOGY (John Libbey 1987), and Ostro et al., American J. Hosp. Pharm. 46: 1576 (1989).

Liposomes can adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents. Scherphof et al., Ann. N.Y. Acad. Sci. 446: 368 (1985). Irrespective of the mechanism or delivery, however, the result is the intracellular disposition of the associated therapeutic.

Anionic liposomal vectors have also been examined. These include pH sensitive liposomes which disrupt or fuse with the endosomal membrane following endocytosis and endosome acidification.

Among liposome vectors, cationic liposomes are the most studied, due to their effectiveness in mediating mammalian cell transfection in vitro. They are often used for delivery of nucleic acids, but can be used for delivery of other therapeutics, be they drugs or hormones.

Liposomes are preferentially phagocytosed into the reticuloendothelial system. However, the reticuloendothelial system can be circumvented by several methods including saturation with large doses of liposome particles, or selective macrophage inactivation by pharmacological means. Classen et al., Biochim. Biophys. Acta 802: 428 (1984). In addition, incorporation of glycolipid- or polyethylene glycol-derivatised phospholipids into liposome membranes has been shown to result in a significantly reduced uptake by the reticuloendothelial system. Allen et al., Biochim. Biophys. Acta 1068: 133 (1991); Allen et al., Biochim. Biophys. Acta 1150: 9 (1993).

Cationic liposome preparations can be made by conventional methodologies. See, for example, Felgner et al., Proc. Nat'l Acad. Sci USA 84:7413 (1987); Schreier, J. of Liposome Res. 2:145 (1992); Chang et al. (1988), supra. Commercial preparations, such as Lipofectin z,900 (Life Technologies, Inc., Gaithersburg, Md. USA), also are available. The amount of liposomes and the amount of DNA can be optimized for each cell type based on a dose response curve. Felgner et al., supra. For some recent reviews on methods employed see Wassef et al., Immunomethods 4: 217-222 (1994) and Weiner, A. L., Immunomethods 4: 217-222 (1994).

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. See COLLOIDAL DRUG DELIVERY SYSTEMS, vol. 66 (J. Kreuter, ed., Marcel Dekker, Inc. 1994).

### Therapeutic Methods of the Invention

The inventive therapeutic methods generally utilize the specific compounds identified above as inhibitors of MUC-1 expression, transport, and/or function. Those agents all share the ability to inhibit MUC-1 function at one or more levels, thus preventing or reducing MUC-1-mediated up-regulation of the T-cell response and/or inducing anergy/immunosuppression. Overall, those compounds will have an immunosuppressive effect.

A typical method, accordingly, involves inducing T-cell-based immnunosuppression or preventing MUC-1-mediated T-cell activation. These methods generally entail contacting a T-cell with an agent that inhibits MUC-1 function. As set out above, these inhibitors affect processes such as MUC-1 transcription, MUC-1 translation, MUC-1 protein transport and/or MUC-1 function inside the T-cell.

Therapeutic methods involve administering to a subject in need of treatment a therapeutically effective amount of an inhibitor, as described above. Some methods contemplate combination therapy with at least one intracellular MUC-1 inhibitor, in conjunction with at least one other immunostimulatory medicament, which may be another MUC-1 inhibitor. The patient may be a human or non-human animal. A patient typically will be in need of treatment when suffering from a disorder associated with MUC-1-induced anergy/immunosuppression or unwanted or illegitimate T-cell down-regulation.

The inventive methods may be employed in vivo or ex vivo. In a typical ex vivo method, for example, peripheral T-cells may be isolated from patients, treated with at least one MUC-1 inhibitor, alone or in combination, and re-infused into the patient.

Administration during in vivo treatment may be by any number of routes, including parenteral and oral, but preferably parenteral. Intracapsular, intravenous, intrathecal, and intraperitoneal routes of administration of MUC-1 and its derivatives may be employed. The skilled artisan will recognize that the route of administration will vary depending on the disorder to be treated. For example, intracapsular administration may be used when treating arthritis. Injection into the hepatic portal vein may be employed when treating inflammatory hepatitis. Intra-organ injection of the thyroid may be used when treating thyroiditis.

Either intravenous or intraperitoneal administration may be used when treating autoimmune diseases of the gastrointestinal tract, such as pancreatitis or colitis. Intrathecal administration may be appropriate when treating autoimmune encephalitis.

Intravenous or intra-organ injections may be employed to prevent or suppress transplant rejections, such as kidney transplants.

Intracellular MUC-1 inhibitors may be administered alone, in combination with each other, or in combination with other medicaments. Ideally these other medicament agents will be immunomodulators, and preferably will have immunosuppressant properties. Both protein and non-protein agents are contemplated. Intracellular MUC-1 inhibitors may be co-administered with conventional immunosuppressants.

Determining a pharmaceutically effective amount of intracellular MUC-1 inhibitor is well within the purview of the skilled clinician, and largely will depend on the exact identity of the inhibitor, particular patient characteristics, route of administration and the nature of the disorder being treated. General guidance can be found, for example, in the publications of the International Conference on Harmonisation and in REMINGTON'S PHARMACEUTICAL SCIENCES, chapters 27 and 28, pp. 484-528 (Mack Publishing Company 1990).

Determining a pharmaceutically effective amount specifically will depend on such factors as toxicity and efficacy of the medicament. Toxicity may be determined using methods well known in the art and found in the foregoing references. Efficacy may be determined utilizing the same guidance in conjunction with the methods described below in the Examples. A pharmaceutically effective amount, therefore, is an amount that is deemed by the clinician to be toxicologically tolerable, yet efficacious. Efficacy, for example, is measured by induction or substantial induction of anergy/immunosuppression or substantial alleviation of an unwanted/illegitimate T-cell activation, in accord with the definition of “treating” discussed above.

The foregoing discussion and following examples are presented merely for illustrative purposes and are not meant to be limiting. Thus, one skilled in the art will readily recognize additional embodiments within the scope of the invention that are not specifically exemplified.

### EXAMPLES

### Example 1

### Materials and Methods

A. Antibodies/reagents

Mouse IgG, goat IgG and MOPC.21 (IgG1), were obtained from Sigma (Mississauga, Ontario, Canada). The cell culture media RPMI-1640, fetal bovine serum (FBS) and AIM V were obtained from Gibco BRL (Burlington, Ontario, Canada). Anti-CD3-FITC, anti-CD4-FITC/CD8-PE, IgG1-FITC/IgG1-PE, leukogate (CD45-FITC/CD14-PE), IgG1-FITC/IgG2-PE simultest control, anti-CD25-PE and anti-CD69-PE were purchased from Becton & Dickinson (San Jose, Calif., USA). Goat anti-mouse IgG1-PE, IgG1-FITC and isotype control mouse IgG1 were obtained from Southern Biotech (Birmingham, Ala., USA). Ficoll-Hypaque was obtained from Pharmacia Biotech (Baie d'Urfe, Quebec, Canada). Anti-CD3 (OKT3) was used as purified antibody obtained from culture supernatant of clones purchased from American Type Culture Collection (ATCC; Rockville, Md., USA). Anti-human-MUC-1 mAb B27.29 was purified from culture supernatant of the cell line B27.29 (Reddish et al., 1992 J. Tumor Marker Oncol. 7:19).

B. Cell Surface Immunofluorescence Staining

Peripheral blood lymphocytes (PBLs) were isolated from buffy coats obtained from normal healthy donors (Canadian Red Cross, Edmonton, Alberta, Canada). For detection of cell surface antigens, PBLs cultured as indicated in each experiment were stained essentially as previously described (Agrawal et al., J. Immunol. 157:3229 (1996). Anti-MUC-1 mAb B27.29 (2 μg/5×10⁵ T-cells) or isotype control antibody B80.3 (2 μg/5×10⁵ T-cells) were used with indirect labelling with FITC or PE conjugated second antibody (GαM IgG1). In parallel, appropriate isotype control antibody was always used to stain the cells in a similar way. The isotype control groups had <2% positive cells. The samples were analyzed by flow cytometry using FACSort® (Becton & Dickinson). Percent positive cells were defined as the fraction of cells exhibiting fluorescence intensities beyond a region set to exclude at least 98% of the control isotype matched antibody stained cells.

C. Proliferation Assay

PBLs were stimulated with PHA (1 μg/ml) for 3 days, T-cells were then harvested and recultured in the presence or absence of OKT3, B27.29 mAb, isotype control mAb B80.3 and Goat anti-mouse in 96 well plates in quadruplicate. On the third day, the wells were pulsed with 1 uCi/well ³H Thymidine (Amersham). Incorporation of ³H Thymidine into DNA of proliferating T-cells was measured after harvesting the plates after 18-24 h and counting in liquid scintillation counter (Beckman LS 60001C, Mississauga, ON, Canada).

D. Determination of mRNA for Human MUC-1 by PCR

MUC-1 mRNA in the lymphocytes was analyzed using reverse transcription PCR (RT-PCR). Total RNA was extracted from the T-cells using Trizol according to manufacturer's instructions (Life Technologies) and was reverse transcribed into cDNA with M-MLV Reverse Transcriptase and oligo d(T) (Perkin Elmer, Norwalk, Conn.). Subsequent DNA amplification was performed in the same tubes using AmpliTaq DNA polymerase (Perkin Elmer, Norwalk, Conn.) and MUC-1 specific primers (5′-TCTACTCTGGTGCACAACGG-3′ and 5′-TTATATCGAGAGGCTGCTTCC-3′). These primers spanned a region within the genomic DNA that contained 2 introns and would result in the amplification of a 489 bp fragment from RNA and a 738 bp fragment from any contaminating genomic DNA. MCF-7 (human breast cancer cell line obtained from ATCC) RNA was used as a positive control and mouse spleen RNA was used as a negative control. RNA specific primers for human beta actin were used as a positive control with each RNA sample. Amplified fragments were run on a 2% agarose gel. All samples from lymphocytes that had been stimulated with PHA, produced a fragment of approximately 489 bp indicating the presence of human MUC-1 mRNA. Samples from unstimulated lymphocytes produced either no fragment or a faint product upon gel electrophoresis indicating no MUC-1 message or only a small amount.

E. Determination of Soluble MUC-1 Mucin in Cell Supernatants

MUC-1 in cell culture supernatants was determined with a sandwich enzyme immunoassay (EIA) employing mAb B27.29 (Biomira Inc.) as solid phase on polystyrene microwells (Nunc Maxisorp™), horseradish peroxidase (HRP, Boehringer Mannheim), conjugated mAb B27.29 as tracer, and tetramethylbenzidine (TMB, Biomira Diagnostics Inc., Toronto, Ontario, Canada) as substrate. The HRP-B27.29 conjugate was prepared with the heterobifunctional cross-linker Sulfo-SMCC (Pierce). The EIA was calibrated by correlation with the TRUQUANT® BR™ RIA (Biomira Diagnostics Inc.). Cell culture supernatants were assayed undiluted; under these conditions the lower limit of detection is estimated to be in the range of 0.01-0.02 Units/ml.

### Example 2

### MUC-1 Mucin is Expressed on the Surface of Mitogen Activated Human T-cells

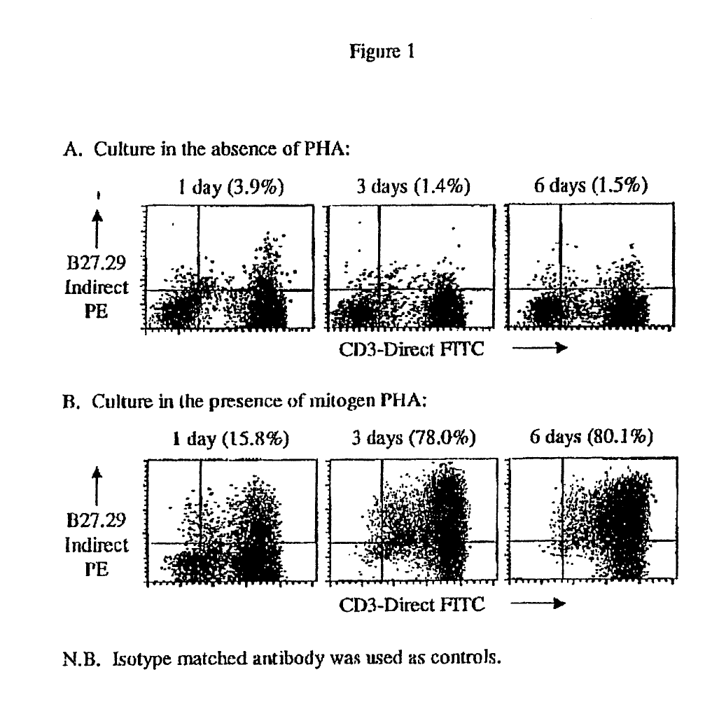

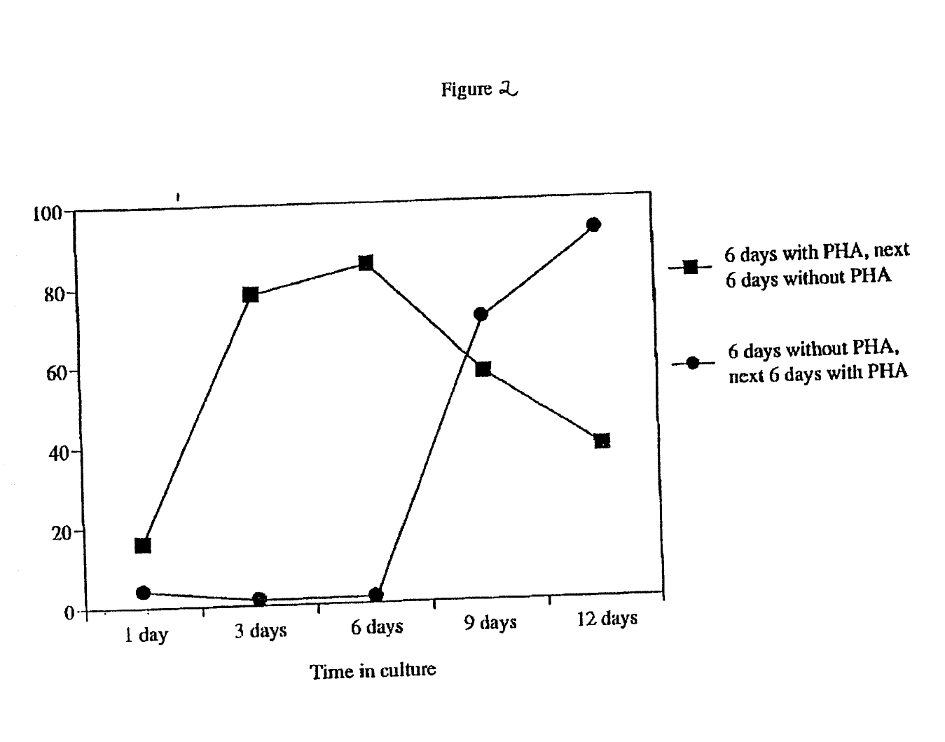

PBLs obtained from buffy coats of normal Red Cross donors were stimulated with PHA for various time periods. Expression of MUC-1 mucin on the surface of PHA activated T-cells was examined by flow cytometry using anti-MUC-1 monoclonal antibody B27.29. MUC-1 mucin expression was examined at 1 day, 3 days and 6 days after in vitro culture initiation with or without PHA stimulation. FIG. 1, presents the time course of MUC-1 expression on activated human T-cells. At each time point, cells were collected and stained for CD3 and MUC-1 expression. The top (A) row represents cells in the absence of mitogen stimulus and the bottom (B) row represents cells cultured in the presence of PHA. As controls isotype matched antibody was used (data not shown), that stained <2% of the cells. The number in parentheses represents percent MUC-1 positive T-cells.

FIG. 1 demonstrates that in cultures without added PHA there was a low (1-4%) number of MUC-1 positive cells in the CD3⁺ T-cell population. In PHA stimulated cultures there was an increase in the number of B27.29⁺ CD3⁺ cells to a peak of approximately 80% positive cells 3 to 6 days post culture initiation. As a control for mAb B27.29 binding specificity, we determined whether the presence of soluble MUC-1 mucin inhibits mAb B27.29 binding to 3 day PHA activated T-cells. We observed a MUC-1 mucin dose dependent inhibition of staining of activated human T-cells with B27.29: at 1 μg of MUC-1 mucin, a 25% inhibition of binding was noted, at 10 μg MUC-1 a 45% inhibition and at 50 μg MUC-1 a 65% inhibition of B27.29 binding to activated T-cells was noted. A negative control mucin (OSM) did not inhibit binding of B27.29 to PHA activated T-cells (0% inhibition of binding of mAb B27.29 at 50 μg OSM).

### Example 3

### mRNA for MUC-1 Mucin is Present in Activated T-cells

In order to confirm that the appearance of cell surface MUC-1 on activated T-cells represents the presence of newly synthesized mucin, RT-PCR was performed in a time course experiment where the expression of MUC-1 on the cell surface was determined at the same time as MUC-1 mRNA determination. Both MUC-1 mRNA and surface expression were determined in T-cells cultured in the presence or absence of PHA after 1 day, 3 day and 6 days after culture. Gel electrophoresis demonstrated that MUC-1 specific mRNA could be detected by RT-PCR after 24 h of PHA stimulation with increased expression noted at days 3 and 6. MUC-1 mRNA was present in PHA stimulated cells but not in the unstimulated cells and correlated with surface expression of MUC-1 (see FIG. 1).

### Example 4

### MUC-1 Mucin is Expressed by Both CD4 and CD8 Positive T-cells

Double staining with anti-CD4 or anti-CD8 mAbs and mAb B27.29 demonstrates that at days 5 and 7 after activation of PBLs with PHA, approximately 80% of the CD4⁺ T-cells are MUC-1 positive and approximately 65% of the CD8⁺ T-cells are MUC-1 positive (Table I).

TABLE I
MUC-1 is expressed on both CD4⁺ and CD8⁺ T-cells

| Time after PHA | | % of CD4⁺ T-cells | % of CD8⁺ T-cells |
|---|---|---|---|
| Stimulation | | positive for MUC-1 | positive for MUC-1 |
| 5 | days | 86.6% | 69.6% |
| 7 | days | 80.7% | 66.5% |

### Example 5

### MUC-1 Mucin is Co-expressed with Other T-cell Activation Markers

Double staining for MUC-1 mucin expression with anti-CD25 or anti-CD69 mAbs was carried out on days 1, 3, 6 following T-cell activation with PHA. Table II demonstrates that the percentage of cells co-expressing CD69 or CD25 and MUC-1 mucin increased with time in culture. However, the kinetics of CD69 or CD25 expression seems to be different than that of MUC-1 expression because at day 1 after stimulation approximately 18% of the CD25⁺ T-cells are MUC-1 positive and 15% of the CD69⁺ T-cells are MUC-1 positive; at day 3 after stimulation approximately 74% of the CD25⁺ T-cells are MUC-1 positive and 75% of the CD69⁺ T-cells are positive; finally, at day 6 after stimulation approximately 80% of both CD25⁺ and CD69⁺ T-cells are MUC-1 positive.

TABLE II
MUC-1 coexpressed with other T-celI activation markers

| Time after PHA | | % of CD69⁺ T-cells | % of CD25⁺ T-cells |
|---|---|---|---|
| stimulation | | positive for MUC-1 | positive for MUC-1 |
| 1 | h | 9.1 | ND |
| 4 | h | 8.1 | ND |
| 1 | day | 14.75% | 17.4% |
| 3 | days | 75.5% | 74.3% |
| 6 | days | 81.6% | 80.2% |

### Example 6

### Down Regulation of MUC-1 Expression on Activated T-cells Following Removal of the Mitogen

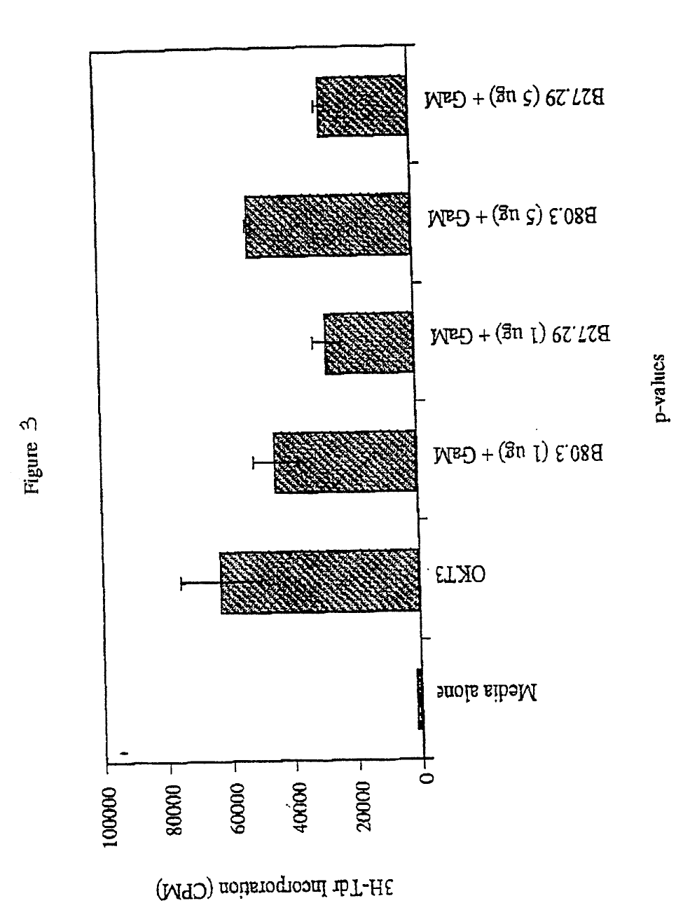

T-cells were cultured in the presence of PHA for 1, 3 and 6 days, followed by washing and reculturing in media without PHA for an additional 3 and 6 days. FIG. 3 shows that expression of MUC-1 mucin on T-cells is reversible. (n) PBLs were cultured in the presence of PHA for 1, 3 and 6 days. At day 6, the cells were washed, harvested and recultured in the absence of PHA (media alone) for further 3-6 days. (I) PBLs were cultured in the absence of PHA for 6 days after which PHA was added and cells were cultured again for a further 6 days. In both groups, cells were harvested at each time point 1, 3, 6, 9 and 12 days and double stained for CD3 and MUC-1 (B27.29 mAb) expression. Data is shown as the mean percent of MUC-1 positive T-cells±S.D.

As shown in FIG. 3, after removing the PHA from the cultures, MUC-1 expression was reduced with time. This reduction in MUC-1 expression is analogous to transient expression of T-cell activation marker CD69 (Testi et al., J. Immunol. 142: 1854-1860 (1989)). It was found that surface CD69 expression reaches to peak level by 18-24 h after stimulation and declines with the removal of stimuli. In addition, T-cells cultured in the absence of PHA for 1, 3 and 6 days and then stimulated with PHA, MUC-1 on T-cells expression was not observed up to 6 days in culture without PHA but MUC-1 expression is apparent after subsequent

### Example 7

### Soluble MUC-1 Mucin is Found in Cell Supernatants of Activated Human T-cell Cultures

An enzyme-linked immunoassay (EIA) specific for MUC-1 mucin was used to test supernatants from PHA activated T-cells for the presence of soluble MUC-1 mucin. Table III shows that supernatants from PHA activated but not non-activated cultures contained increasing amount of soluble MUC-1 mucin with a peak level of approximately 27 U/ml culture supernatant at day 6.

TABLE III
Activated human T-cells secrete or shed detectable amounts of MUC-1
into culture supernatants

| | | Amount of secreted MUC-1 (U/ml × 10²) mean ± S.D. | |
|---|---|---|---|
| Time in Culture | | PBLs without PHA | PBLs with PHA (1 ug/ml) |
| 1 | day | 2.0 ± 0.1 | 1.5 ± 0.2 |
| 3 | days | 1.6 ± 0.1 | 12.9 ± 1.0 |
| 6 | days | 1.3 ± 0.0 | 27.2 ± 3.6 |
| 7 | days | 1.7 ± 0.1 | 24.2 ± 0.1 |

### Example 8

### Cross-linking Surface MUC-1 Mucin by Antibody Modulates T-cell Proliferative Response

Human PBLs were stimulated with PHA for 3 days to induce the expression of MUC-1 mucin. At this time the cells were harvested, washed, and recultured in the presence of anti-CD3 (OKT3, as polyclonal stimuli), with or without anti-MUC-1 mAb B27.29 and Goat-anti-mouse antibody. It appears that the T-cells stimulated in the presence of MUC-1 cross-linking conditions, the proliferation response was lower than that of the cells cultured in the presence of isotype control antibody. This experiment is illustrated in FIG. 4. There, human PBLs were cultured in the presence of PHA for 3 days. At this time, cells were harvested and set up in 96 well flat bottom plate at 1×10⁵ cells/well in the presence or absence of media, OKT3 (αCD3 as stimulant), αMUC-1 (B27.29 mAb) and Goat-anti-mouse antibody. On the third day of culture, ³H-Tdr was added and proliferation was measured on the fourth day. The data represent mean CPM±S.D. of four replicate wells.

### Example 9

### MUC-1 Antisense Molecules Prevent T-cell Activation

This example shows that the inhibition of MUC-1 intracellularly prevents T-cell activation. Antisense oligodeoxynucleotides were obtained from Chemicon (Biodiagnostics). Control sequences were directed to carcinoembryonic antigen (CEA) and epidermal growth factor (EGF), both irrelevant to T-cell activation. Antisense molecules had the following sequences:

TABLE IV
MUC-1 Antisense Sequences

| | MUC-1 |
|---|---|
| SEQUENCE (IDENTIFIER) | COORDINATES* |
| GGTGTCATGGTGGTGGTGAAA (D02) | 61-81 |
| AGACTGGGTGCCCGGTGTCAT (D03) | 74-94 |
| GCAGGAAGAAAGGAGACTGGG (D04) | 87-107 |
| TAGAGCTTGCATGACCAGAA (D05) | 141-161 |
| CGGGGCTGAGGTGACATCGT (D06) | 419-439 |
| ATCTCGAACGTACTGGTCTTG (D07) | Complement of |
| | 141-161 |

*Coordinates are relative to Genebank Accession Number J05582, which is the mRNA for human MUC-1. The ATG start site is at position 74, and the complement of the start codon is indicated above with underlining.

Purified human peripheral T lymphocytes were plated, 2×10⁵ cells per well. Cells were stimulated with 0.2 μg of PHA per well, in the presence or absence of antisense molecules at 1 nM and 2.5 nM. The antisense molecules were delivered using LIPOFECTIN (Life Technologies), as described in the accompanying instructions (Form No. 187057M), except that 1.5 μl per well, rather than 2-25 μl, of LIPOFECTIN Reagent was used. After 72 hours cells were harvested washed and subjected to FACS analysis for surface MUC-1, CD3 and CD25. Control oligos were GCCGAGGTGACACCGTGGGCTG (B02) and CGGCYCCACTGGCACCCGAC (B03), which correspond to a MUC-1 tandem repeat sequence and an inverted MUC-1 tandem repeat sequence, respectively.

FACS analysis, using antibodies to CD3, CD25 and MUC-1 indicated that the expression of both MUC-1 and CD25 were inhibited, but CD3 was not. In addition, qualitatively the sizes of the cells more closely resembled small, resting (non-activated) T-cells. The controls yielded the opposite result.

Cell proliferation assays were done in triplicate. Briefly 2×10⁵ cells were plated per well as above for the FACS analysis, with PHA and varying amounts of MUC-1 antisense or control molecules. Incorporation of tritiated thymidine was measured on day 4, following an overnight pulse with 1 μCi/well. On such assay is exemplified in Table V.

TABLE V
Cell Proliferation Assay

| SAMPLE | | AVERAGE CPM | STANDARD DEVIATION |
|---|---|---|---|
| Cells Only | | 364 | 2 |
| Add PHA | | 34172 | 2725 |
| Add Lipofectin | | 66281 | 2553 |
| 2 | nmol D02 | 793 | 696 |
| 2 | nmol D03 | 534 | 108 |
| 1 | nmol D04 | 28213 | 5005 |
| 2 | nmol D05 | 324 | 194 |
| 2 | nmol D06 | 1208 | 213 |
| 2 | nmol D07 | 943 | 68 |
| 2 | nmol B02 | 69745 | 14300 |
| 2 | nmol B03 | 148366 | 17411 |

Table V shows that most of the MUC-1 antisense molecules tested inhibited PHA-mediated T-cell proliferation with D02, D03, and D05-D07 being particularly effective. Thus, the cell proliferation and FACS data both indicate that the intracellular inhibition of MUC-1 prevents T-cell activation.

The foregoing detailed discussion and working examples are presented merely for illustrative purposes and are not meant to be limiting. Thus, one skilled in the art will readily recognize additional embodiments within the scope of the invention that are not specifically exemplified.

What is claimed is:

1. A method of inducing T-cell-based immunosuppression, comprising contacting a T-cell with an agent that acts inside the cell to inhibit a cellular process selected from the group consisting of MUC-1 function, MUC-1 transcription, MUC-1 translation and MUC-1 protein transport.

2. A method according to claim 1, wherein said agent comprises a polynucleotide which is complementary to a portion of the 5′ end of the MUC-1 mRNA.

3. A method according to claim 2, wherein said polynucleotide is complementary to a portion of the MUC-1 mRNA within about 25 nucleotides of the MUC-1 start codon.

4. A method according to claim 1, wherein said agent is associated with a cell surface targeting sequence.

5. A method according to claim 1, wherein said targeting sequence is an RGD targeting sequence.

6. A method of treating an immune disorder, comprising administering to a patient in need of treatment an agent that intracellularly inhibits a cellular process selected from the group consisting of MUC-1 function, MUC-1 transcription, MUC-1 translation and MUC-1 protein transport.

7. A method according to claim 6, wherein said immune disorder is selected from the group consisting of transplant rejection, an autoimmune disorder and an inflammatory disorder.

8. A method according to claim 7, wherein said autoimmune disorder is selected from the group consisting of myasthenia gravis, systemic lupus erythematosus, polyarteritis nodosa, Goodpastures syndrome, isopathic thrombocytopenic purpura, autoimmune hemolytic anemia, Grave's disease, rheumatic fever, pernicious anemia, insulin-resistant diabetes mellitus, bullous pemphigold, pemphigus vulgaris, viral myocarditis (Cocksakie B virus response), autoimmune thyroiditis (Hashimoto's disease), male infertility (autoimmune), sarcoidosis, allergic encephalomyelitis, multiple sclerosis, Sjorgens disease, Reiter's disease, Celiac disease, sympathetic ophthalmia, and primary biliary cirrhosis.

9. A method according to claim 7, wherein said inflammatory disorder is selected from the group consisting of inflammatory arthritis, psoriasis, allergies, and ankylosing spondylitis.

10. An intracellular antagonist of MUC-1, comprising an antagonist of MUC-1 function associated with a domain selected from the group consisting of a targeting domain, an internalization domain and combinations thereof.

11. An antagonist according to claim 10, wherein said targeting domain is an RGD targeting sequence.

12. An antagonist according to claim 10, wherein said antagonist of MUC-1 function comprises from one to three MUC-1 core repeats.

13. An antagonist according to claim 10, wherein said antagonist of MUC-1 function is a fragment of an antibody directed to MUC-1.

14. An antagonist according to claim 10, wherein said antagonist of MUC-1 function is an antisense nucleic acid molecule.

15. An antagonist according to claim 10, further comprising a retrograde transport sequence or a tat protein translocation domain.

16. An antagonist according to claim 10, in combination with an immunosuppressant.

17. A composition according to claim 16, wherein said immunosuppressant is selected from the group consisting of azathioprine, chlorambucil, cyclophosphamide, cyclosporine, dactinomycin, methotrexate and thioguanine, dexamethasome, betamethasone, cortisone, hydrocortisone, mycophenolate, and prednisolone.

18. An antisense oligonucleotide that is complementary to the 5′ end of the MUC-1 mRNA.

19. An antisense oligonucleotide that is complementary to a portion of the MUC-1 mRNA within about 25 nucleotides of the MUC-1 start codon.
